(19) United States
(12) Patent Application Publication
(10) Pub. No.: US 2010/0185256 A1
(43) Pub. Date: Jul. 22, 2010

(54) METHODS AND SYSTEMS FOR ESTABLISHING, ADJUSTING, AND/OR MODULATING PARAMETERS FOR NEURAL STIMULATION BASED ON FUNCTIONAL AND/OR STRUCTURAL MEASUREMENTS

(75) Inventor: Justin Hulvershorn, Seattle, WA (US)

Correspondence Address: St. Jude Medical Neuromodulation Division, 6901 Preston Road, Plano, TX, 75024, US

(73) Assignee: Northstar Neuroscience, Inc., Seattle, WA (US)

(21) Appl. No.: 12/355,727

(22) Filed: Jan. 16, 2009

(51) Int. Cl. A61N 1/36

(52) U.S. Cl. 607/45

(57) ABSTRACT

Methods and systems for establishing, adjusting, and/or modulating parameters for neural stimulation based, at least in part, on functional and/or structural measurements are disclosed. A method in accordance with one embodiment includes measuring a volume of functionally active neural tissue within a patient's central nervous system both before and after affecting a target neural population of the patient with electromagnetic stimulation. The method further includes controlling at least one signal delivery parameter with which the electromagnetic stimulation is applied to the patient based, at least in part, on the measured difference in the volume of functionally active neural tissue.

### TECHNICAL FIELD

The present disclosure is directed generally toward methods and systems for establishing, adjusting, and/or modulating parameters for neural stimulation including, but not limited to, techniques for determining signal delivery parameters based, at least in part, on functional and/or structural measurements.

### BACKGROUND

A wide variety of mental and physical processes are controlled or influenced by neural activity in particular regions of the brain. In some areas of the brain, such as in the sensory or motor cortices, the organization of the brain resembles a map of the human body; this is referred to as the “somatotopic organization of the brain.” There are several other areas of the brain that appear to have distinct functions that are located in specific regions of the brain in most individuals. For example, areas of the occipital lobes relate to vision, regions of the left inferior frontal lobes relate to language in the majority of people, and regions of the cerebral cortex appear to be consistently involved with conscious awareness, memory, and intellect. This type of location-specific functional organization of the brain, in which discrete locations of the brain are statistically likely to control particular mental or physical functions in normal individuals, is herein referred to as the “functional organization of the brain.”

Many problems or abnormalities with body functions can be caused by damage, disease, and/or disorders in the brain. A stroke, for example, is one very common condition that damages the brain. Strokes are generally caused by emboli (e.g., obstruction of a vessel), hemorrhages (e.g., rupture of a vessel), or thrombi (e.g., clotting) in the vascular system of a specific region of the cortex, which in turn generally causes a loss or impairment of a neural function (e.g., neural functions related to face muscles, limbs, speech, etc.). Stroke patients are typically treated using physical therapy to rehabilitate the loss of function of a limb or another affected body part. Stroke patients may also be treated using physical therapy plus an adjunctive therapy, such as amphetamine treatment. For most patients, however, such treatments are minimally effective and little can be done to improve the function of an affected body part beyond the recovery that occurs naturally without intervention. As a result, many types of physical and/or cognitive deficits that remain after treating neurological damage or disorders are typically considered permanent conditions that patients must manage for the remainder of their lives.

Neurological problems or abnormalities are often related to electrical and/or chemical activity in the brain. Neural activity is governed by electrical impulses or “action potentials” generated in neurons and propagated along synaptically connected neurons. When a neuron is in a quiescent state, it is polarized negatively and exhibits a resting membrane potential typically between −70 and −60 mV. Through chemical connections known as synapses, any given neuron receives excitatory and inhibitory input signals or stimuli from other neurons. A neuron integrates the excitatory and inhibitory input signals it receives, and generates or fires a series of action potentials when the integration exceeds a threshold potential. A neural firing threshold, for example, may be approximately −55 mV.

It follows that neural activity in the brain can be influenced by electrical energy supplied from an external source such as a waveform generator. Various neural functions can be promoted or disrupted by applying an electrical current to the cortex or other region of the brain. As a result, researchers have attempted to treat physical damage, disease and disorders in the brain using electrical or magnetic stimulation signals to control or affect brain functions.

Transcranial electrical stimulation (TES) is one such approach that involves placing an electrode on the exterior of the scalp and delivering an electrical current to the brain through the scalp and skull. Another treatment approach, transcranial magnetic stimulation (TMS), involves producing a magnetic field adjacent to the exterior of the scalp over an area of the cortex. Yet another treatment approach involves direct electrical stimulation of neural tissue using implanted electrodes.

The neural stimulation signals used by these approaches may comprise a series of electrical or magnetic pulses that can affect neurons within a target neural population. Stimulation signals may be defined or described in accordance with stimulation signal parameters, including pulse amplitude, pulse frequency, duty cycle, stimulation signal duration, and/or other parameters. Electrical or magnetic stimulation signals applied to a population of neurons can depolarize neurons within the population toward their threshold potentials. Depending upon stimulation signal parameters, this depolarization can cause neurons to generate or fire action potentials. Neural stimulation that elicits or induces action potentials in a functionally significant proportion of the neural population to which the stimulation is applied is referred to as supra-threshold stimulation; neural stimulation that fails to elicit action potentials in a functionally significant proportion of the neural population is referred to as sub-threshold stimulation. In general, supra-threshold stimulation of a neural population triggers or activates one or more functions associated with the neural population, but sub-threshold stimulation by itself does not trigger or activate such functions. Supra-threshold neural stimulation can induce various types of measurable or monitorable responses in a patient. For example, supra-threshold stimulation applied to a patient's motor cortex can induce muscle fiber contractions in an associated part of the body.

More recently, direct cortical stimulation has been used to produce therapeutic, rehabilitative, and/or restorative neural activity, as disclosed in U.S. Pat. No. 7,010,351 and pending U.S. patent application Ser. No. 10/606,202, both assigned to the assignee of the present application, and both incorporated herein by reference. These techniques have been used to produce long lasting benefits to patients suffering from a variety of neural disorders. While these techniques have been efficacious, there is a continued need to improve the applicability of these methods to a wide variety of patients, and to further enhance the longevity of the effects produced by these methods.

### BRIEF DESCRIPTION OF THE DRAWINGS

FIG. 1A is a schematic view of neurons.

FIG. 1B is a graph illustrating firing an “action potential” associated with normal neural activity.

FIG. 2 is a flow diagram illustrating a process for treating a patient in accordance with an embodiment of the invention.

FIG. 3 is a top plan image of a portion of a brain illustrating neural activity in a first region of the brain associated with the neural function of the patient according to the somatotopic organization of the brain.

FIGS. 4A and 4B are top plan images of a portion of the brain of FIG. 3 illustrating a loss of neural activity associated with the neural function of the patient used in one stage of a method in accordance with an embodiment of the invention.

FIG. 5 is a top plan image of the brain of FIG. 3 showing a change in location of the neural activity associated with the neural function of the patient at another stage of a method in accordance with an embodiment of the invention.

FIG. 6 is a flow diagram illustrating a process for treating a patient in accordance with an embodiment of the invention.

FIG. 7A is a top plan image of a portion of a first brain map and a portion of a second brain map illustrating a loss of neural activity associated with the neural functions of control group patients and investigational group patients, respectively, used in one stage of a method in accordance with an embodiment of the invention.

FIG. 7B is a top plan image of the first and second brain maps of FIG. 7A showing changes in the volume and location of the neural activity associated with the neural function of the patients at another stage of a method in accordance with an embodiment of the invention.

FIGS. 8A and 8B are top plan images of a portion of a brain illustrating a loss of neural activity associated with the neural function of a patient before therapy and a change in volume and location of the neural activity associated with the neural function of the patient after therapy in accordance with another embodiment of the invention.

FIGS. 9A and 9B are top plan images of a portion of a brain illustrating a loss of neural activity associated with the neural function of a patient before therapy and a change in volume and location of the neural activity associated with the neural function of the patient after therapy in accordance with still another embodiment of the invention.

FIG. 10 is a partially schematic illustration of a stimulation device configured in accordance with an embodiment of the invention.

FIG. 11 illustrates a stimulation device operatively coupled to an external controller in accordance with another embodiment of the invention.

FIG. 12 is a schematic illustration of a pulse system configured in accordance with an embodiment of the invention.

FIG. 13 is an isometric illustration of a device that carries electrodes in accordance with another embodiment of the invention.

FIG. 14 is a partially schematic, side elevation view of an electrode configured to deliver electromagnetic stimulation to a subcortical region in accordance with an embodiment of the invention.

FIG. 15 is a partially schematic, isometric illustration of a magnet resonance chamber in which the effects of neural stimulation may be detected and evaluated.

FIG. 16 illustrates a patient wearing a network of electrodes positioned to detect brain activity in accordance with further embodiments of the invention.

### DETAILED DESCRIPTION

### A. Introduction

The following disclosure is directed generally toward methods and systems for establishing, adjusting, and/or modulating signal delivery parameters for neural stimulation based, at least in part, on functional and/or structural measurements of neural activity. Several embodiments of methods and systems described herein, for example, are directed toward enhancing or otherwise inducing neuroplasticity to effectuate a particular neural function. Neuroplasticity refers to the ability of the brain to change or adapt over time. It was once thought adult brains became relatively “hard wired,” such that functionally significant neural networks could not change significantly over time or in response to injury. It has become increasingly more apparent that these neural networks can change and adapt over time so that meaningful function can be regained in response to brain injury.

An aspect of several embodiments of methods and systems in accordance with the disclosure is to use one or more measurements of functional activity during adaptive, restorative, and/or compensatory neuroplasticity to adjust and/or modulate signal delivery parameters for one or more therapy sequences. One particular embodiment can include, for example, using functional neuroimaging (e.g., functional MRI (fMRI)) to detect changes in a volume of functionally activated tissue (e.g., activity level or firing rate) of one or more neural populations of a patient before and after one or more therapy sequences. As described in detail below, a reduction in the volume of functionally activated tissue can indicate that neural activity is tending toward a more normal state. Accordingly, the stimulation parameters in the one or more subsequent therapy sequence can be changed based, at least in part, on the detected volume changes. As discussed in detail below, functional neuroimaging, as well as other suitable functional and/or structural measurements, can provide useful benchmarks or indicators before, during, and/or after treatment, and can be used to guide adjustments and/or modulations in the treatment parameters during each therapy sequence.

Various methods and systems in accordance with embodiments of the disclosure electrically and/or magnetically stimulate the brain at a stimulation site where neuroplasticity is occurring or has occurred, and/or where neuroplasticity is expected to occur. In particular embodiments, the manner in which the electromagnetic signals are applied to the brain and/or other neural tissue can be varied over the course of two or more therapy sequences (e.g., time periods). For example, a type of signal source and/or a waveform, amplitude, pulse pattern, and/or location at which stimulation is applied can be varied from one time period to the next. In still further embodiments, the manner in which one or more adjunctive therapies are applied during a therapy sequence can be varied from one time period to another. For example, a type of behavioral therapy and/or a manner in which a patient undergoes such therapy can be varied. The adjunctive therapy can occur simultaneously with the electromagnetic stimulation, or at other times, depending upon the patient's condition.

The various systems described herein can support different modes via which electromagnetic signals are applied or delivered to the patient. For example, a system in accordance with one embodiment can include a controller that is coupleable to at least two different kinds of signal delivery devices. The controller can provide electromagnetic signals in accordance with different modes, depending upon which device it is coupled to. The signal delivery devices can be selected to include (for example) implanted cortical electrodes, subcortical or deep brain electrodes, cerebellar electrodes, spinal column electrodes, vagal nerve (or other cranial or peripheral nerve) electrodes, transcranial electrodes and/or transcranial magnetic stimulators. In other embodiments, the systems can have other arrangements and/or include different features. Although many examples described of electromagnetic signal delivery described herein are in the context of stimulation, it will be understood that such signals can have a stimulating or inhibiting effect depending on signal delivery locations, signal characteristics, and/or other parameters.

Several embodiments of methods and systems in accordance with the disclosure can be used to treat particular symptoms in patients experiencing neurologic dysfunction arising from neurological damage, neurologic disease, neurodegenerative conditions, neuropsychiatric disorders, neuropsychological (e.g., cognitive or learning) disorders, and/or other conditions. Such neurologic dysfunction and/or conditions may correspond to Parkinson's Disease, essential tremor, Huntington's disease, stroke, traumatic brain injury, Cerebral Palsy, Multiple Sclerosis, a central and/or peripheral pain syndrome or condition, a memory disorder, dementia, Alzheimer's disease, an affective disorder, depression, bipolar disorder, anxiety, obsessive/compulsive disorder, Post Traumatic Stress Disorder (PTSD), an eating disorder, schizophrenia, Tourette's Syndrome, Attention Deficit Disorder, dyslexia, a phobia, an addiction (e.g., alcoholism or substance abuse), autism, epilepsy, a sleep disorder (e.g., sleep apnea), an auditory disorder (e.g., tinnitus or auditory hallucinations), a language disorder, a speech disorder (e.g., stuttering), migraine headaches, and/or one or more other disorders, states, or conditions. In other embodiments identical or at least generally similar methods and systems can be used to enhance the neural functioning of patients who otherwise function at normal or even above normal levels.

As used herein, measurements of functional activity can include techniques that directly measure neural activity (e.g., electroencephalography (EEG), ECOG, Magnetoencephalography (MEG)), techniques that indirectly measure neural activity (e.g., fMRI, MR perfusion, single photon emission computed tomography (SPECT), Positron Emission Tomography (PET), near infra-red spectroscopy (NIRS), optical tomography (OT), MR Spectroscopy, Ultrasound, Laser Doppler measurements of blood flow), and/or other techniques that measure/indicate long-term changes in neural structure/function (e.g., volumetric MRI, morphometric analysis, Diffusion Tensor Imaging (DTI), DWI, perfusion-weighted imaging). Measurements can be taken in real time in order to continuously adjust therapy (e.g., classic closed loop system), or measurements can be taken at larger intervals of time (e.g., fMRI performed every six months to evaluate efficacy of treatment). Adjusting and/or modulating parameters for the therapy sequences can include changing therapeutic parameters (e.g., polarity, pulse width, frequency, amplitude, etc.), a hiatus in delivering therapy, changing the area of the patient being stimulated, changing the type of therapy (e.g., changing from TMS to tDCS or direct cortical stimulation), and/or the addition or combination of multiple therapies (e.g., adding TMS stimulation to ongoing cortical stimulation).

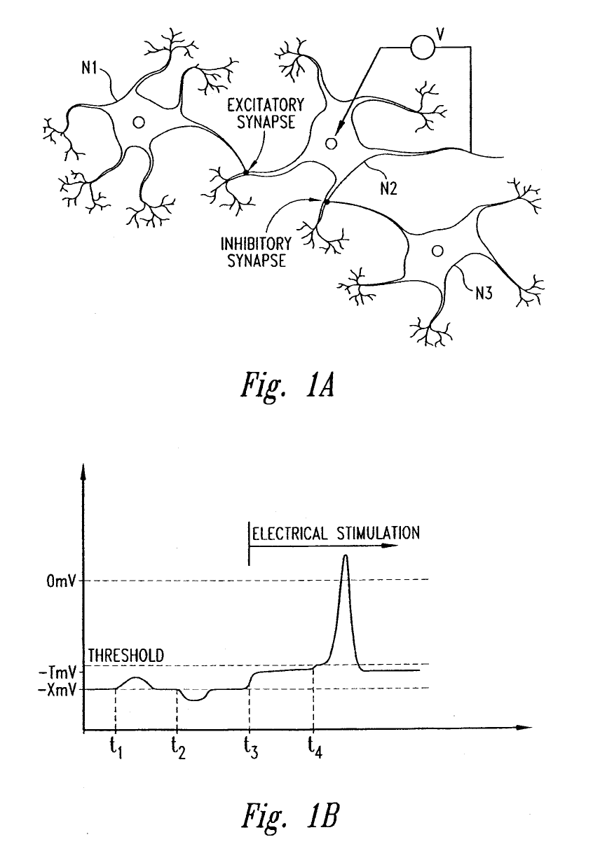

The specific details of certain embodiments of the invention are set forth in the following description and in FIGS. 1A-16 to provide a thorough understanding of these embodiments to a person of ordinary skill in the art. A person skilled in the relevant art will understand that the present invention may have additional embodiments, and that the invention can be practiced without several of the details described below.

B. Methods for Establishing, Adjusting, and/or Modulating Signal Delivery Parameters for Neural Stimulation Based on Functional and/or Structural Measurements

FIG. 1A is a schematic representation of several neurons N1-N3 and FIG. 1B is a graph illustrating an “action potential” related to neural activity in a normal neuron. Neural activity is governed by electrical impulses generated in neurons. For example, neuron N1 can send excitatory inputs to neuron N2 (e.g., at time t₁ in FIG. 1B), and neuron N3 can send inhibitory inputs to neuron N2 (e.g., at time t₂ in FIG. 1B). The neurons receive/send excitatory and inhibitory inputs from/to a population of other neurons. The excitatory and inhibitory inputs can produce “action potentials” in the neurons, which are electrical pulses that travel through neurons by changing the flux of sodium (Na) and potassium (K) ions across the cell membrane. An action potential occurs when the resting membrane potential of the neuron surpasses a threshold level. When this threshold level is reached, an “all-or-nothing” action potential is generated. The action potentials propagate down the length of the axon (the long portion of the neuron that makes up nerves or neuronal tracts) to cause the release of neurotransmitters from that neuron that will further influence adjacent neurons.

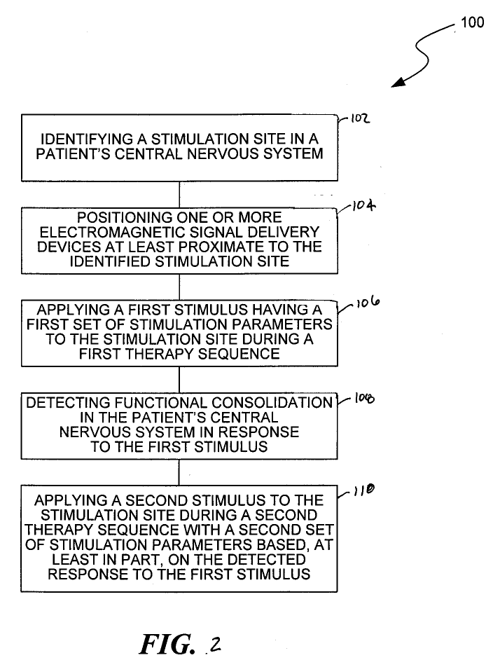

FIG. 2 is a flow diagram illustrating a process 100 for treating a patient in accordance with an embodiment of the invention. The process 100 can include identifying one or more stimulation sites (process portion 102) corresponding to an anatomical region, location, or site at which stimulation (e.g., electromagnetic) signals may be applied or delivered to one or more target neural populations of the patient. In various embodiments, for example, one or more stimulation sites and/or target neural populations may reside upon or within one or more areas of activation and/or deactivation within a specific portion of the patient's brain, central nervous system, or peripheral nervous system. Such signals may be intended to directly and/or indirectly affect the target neural populations by passing or traveling to, into, through, and/or near a target neural population. In particular embodiments, for example, process portion 102 may include identifying one or more anatomical landmarks on the patient that correspond to such neural populations, regions, and/or structures. The anatomical landmarks serve as reference points for identifying or approximately identifying a neural location (e.g., a brain or spinal cord location) where an intended neural activity may occur. Thus, one aspect of the process portion 102 may include referencing a stimulation site relative to anatomical landmarks.

A stimulation site and/or target neural population may be identified and/or located in a variety of manners. For example, a stimulation site and/or target neural population can be identified with one or more procedures involving the identification of anatomical features or landmarks, electrophysiological signal measurement (e.g., EEG, EMG, silent period, coherence, and/or other measurements), neural/neurophysiological imaging (e.g., MRI, fMRI, DTI, PWI, Positron Emission Tomography (PET), and/or SPECT), optical imaging (e.g., NIRS, OT, MEG, and/or another technique), neurofunctional mapping (e.g., using TMS and/or intraoperative stimulation), vascular imaging (e.g., MRA), chemical species analysis (e.g., MRS), and/or another type of functional and/or structural anatomic assessment technique (e.g., TCD). The process portion 102 may additionally (or alternatively) include identifying a stimulation site where neural activity has changed in response to a change in the neural function. In an alternative embodiment, the process portion 102 may include identifying one or more enhanced-precision or patient-specific stimulation sites and/or target neural populations.

In process portion 104, the process 100 can include positioning one or more electromagnetic signal delivery devices or signal transfer elements at least proximate to the identified stimulation site. For example, process portion 104 may include (a) positioning two or more electrodes at a stimulation site (e.g., in a bipolar arrangement); (b) positioning only one electrode at a stimulation site and another electrode remotely from the stimulation site (e.g., in a unipolar arrangement); and/or (c) positioning one or more signal transfer elements transcranially without implanting the signal transfer element(s).

Process portion 106 can include applying a first stimulus having a first set of stimulation parameters to the stimulation site during a first therapy sequence. For example, process portion 106 can include applying an electromagnetic signal to a neural population using a selected current, voltage, and waveform. In process portion 108, the process can include detecting functional consolidation (e.g., using optical techniques, EEG, or by other suitable techniques) in the patient's brain or elsewhere in the patient's central nervous system in response to the first stimulus. As used in this context, “functional consolidation” and “consolidation” refer generally to a reduction in the volume of functionally activated tissue within one or more portions of the patient's central nervous system. In some instances, consolidation can also refer to changes in a level of activation within one or more particular regions of neural tissue (e.g., changes in an intensity level of one or more particular voxels as identified using an fMRI scan). When consolidation occurs, it typically indicates that neural activity is shifting toward a less dysfunctional, more normal state. During therapy, for example, consolidation can be used as an indicator of increased brain normalcy and efficiency. In other embodiments, process portion 108 can additionally include detecting changes in physiologic properties, such as hemodynamic tissue properties (e.g., blood flow levels or blood volume) proximate to the stimulation site, changes in one or more diffusion tracts (e.g., areas of increased fiber density) proximate to the stimulation site, changes in cortical thickness, and/or the number of descending volleys in the spinal cord. In still other embodiments, process portion 108 can include detecting changes in other physiologic properties.

Process portion 110 can include applying a second stimulus to the stimulation site during a second therapy sequence. The second stimulus can be applied with a second set of stimulation parameters based, at least in part, on the detected response to the first stimulus. The process can include, for example, changing, adjusting, and/or modulating the signal delivery mode (e.g., the location to which signals are directed, the type of signal delivery device, the signal parameters including polarity, pulse width, frequency, amplitude, etc., and/or the addition/combination of additional therapies) during the second therapy sequence at process portion 110. However, if it is determined that stimulating the neural population at the stimulation site produces a desired or beneficial result (e.g., consolidation) within the patient's central nervous system, some or all aspects of the second set of stimulation parameters can be selected to be at least approximately identical to the first set of stimulation parameters. Various embodiments of the process 100 are described in greater detail below.

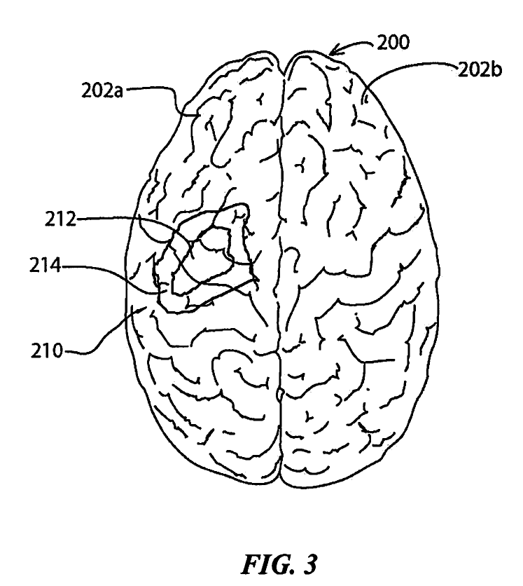

FIGS. 3-5 illustrate specific embodiments of the identification procedure of process portion 102 described above with reference to FIG. 2. As mentioned previously, the process portion 102 can be used to identify one or more regions of the central nervous system where stimulation will likely facilitate or effectuate a desired result, such as rehabilitating a malfunction in or degradation or loss of a neural function caused by a stroke, trauma, disease, or other circumstance. FIG. 3, for example, is a top plan image of a portion of a normal, healthy brain 200 having a first region 210 in a first hemisphere 202a where an intended or normal neural activity occurs to effectuate a specific neural function in accordance with the functional organization of the brain. For example, the neural activity in the first region 210 shown in FIG. 3 is generally associated with the movement of a patient's fingers. The first region 210 can have a high-intensity area 212 and/or a low-intensity area 214 at which different levels of neural activity occur. Although it is not necessary to obtain an image of the neural activity in the first region 210 shown in FIG. 3 to carry out the diagnostic procedure 102, such an image can provide an example of neural activity that typically occurs at a “normal location” according to the functional organization of the brain 200 for a large percentage of people with normal brain function. It will be appreciated that the actual location of the first region 210 will generally vary between individual patients.

The brain 200 also includes a second hemisphere 202b. The two hemispheres 202a and 202b are connected via the corpus callosum, which facilitates information transfer between the hemispheres. Although each hemisphere 202a, 202b generally exerts majority control over motor and/or sensory functions on the opposite or contralateral side of the patient's body, each hemisphere typically also exerts some level of control and/or influence over motor and/or sensory functions on the same or ipsilateral side of the patient's body. Moreover, through transcallosal connections, neural activity in one hemisphere may influence or modulate neural activity (e.g., neuroplasticity, in the opposite hemisphere). The location in the brain 200 that exerts influence on an ipsilateral body function frequently is proximate to or subsumed within the location of the brain associated with a corollary body function. As discussed below, one or more stimulation sites and/or activation sites can be characterized as “ipsilateral” or “contralateral,” with reference to particular brain regions or body functions. In some instances, it may be useful to describe the stimulation sites and/or activation sites with reference to an affected neural population. In such instances, “ipsilesional” is used to refer to a site that is at the same hemisphere as an affected neural population, and “contralesional” is used to refer to a site that is at the opposite hemisphere as the affected neural population, whether the affected neural population is affected by a lesion or another condition. For example, the first region 210 may be associated with a body part or parts (in this example, the fingers of the right hand) and a second region (not shown) in the second hemisphere 202b may be associated with a contralateral homotypic body part (in this case, the fingers of the left hand), i.e., another body part having the same or an analogous structure or function as, but contralateral to, the first body part. This is one example of a body function (movement of the left fingers) that may be a corollary to another body function (movement of the right fingers). Either set of terms may be used herein to characterize the site, depending upon the particular context.

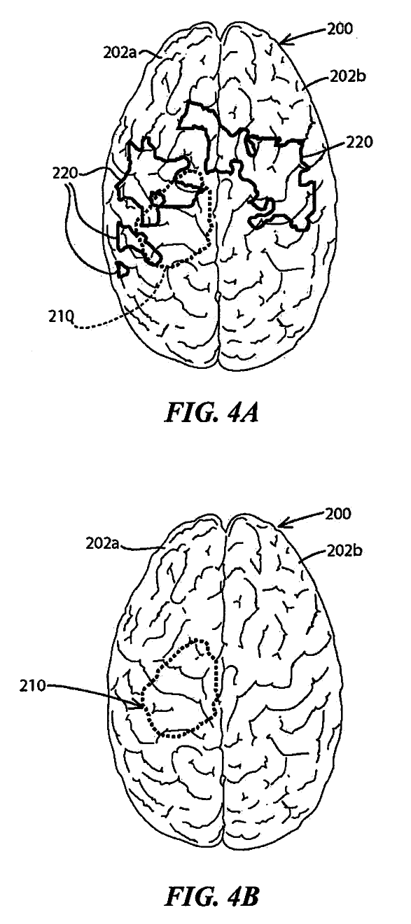

The neural activity in the first region 210, however, can be impaired. In a typical application, the process portion 102 (FIG. 2) may begin by taking an image of the brain 200 that is capable of detecting neural activity to determine whether the intended neural activity associated with the particular neural function of interest is occurring at the region of the brain 200 where it normally occurs according to the functional organization of the brain. FIGS. 4A and 4B, for example, are images of the brain 200 after the first region 210 has been affected (e.g., from a stroke, trauma or other cause). Referring first to FIG. 4A, the neural activity that controlled the neural function for moving the fingers of the right hand no longer occurs in the first region 210 (shown in broken lines). Rather, the neural function or “activation” (as shown by activated sites or regions 220) is dispersed, scattered, or otherwise unconsolidated in both the first hemisphere 202a and the second hemisphere 202b. In some cases, however, the neural activity is diminished rather than being scattered. Referring to FIG. 4B, for example, the image of the brain 200 shows little or no neural activity after the first region 210 (shown in broken lines) has been affected. The first region 210 is thus “inactive” in both the example illustrated in FIG. 4A and the example illustrated in FIG. 4B. This is expected to result in a corresponding loss of the movement and/or sensation in the patient's fingers. In some instances, the damage to the patient's brains 200 may result in only a partial loss of the neural activity in the damaged region. In either case, the images of brain 200 shown in FIGS. 4A and 4B establish that the loss of the neural function is related to the diminished neural activity in the first region 210. The brain 200 may accordingly recruit other neurons to perform neural activity for the affected neural function (e.g., via neuroplasticity), or the neural activity may not be present at any location in the brain.

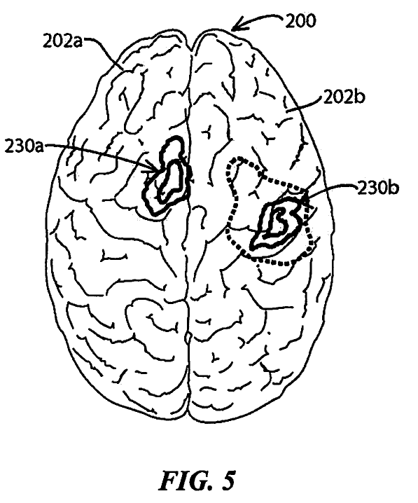

FIG. 5 is an image of the brain 200 illustrating a plurality of potential stimulation sites 230a and 230b for effectuating the neural function that was originally performed in the first region 210 shown in FIG. 3. It is worth noting that the first potential stimulation site 230a is in the same hemisphere 202a as the first region 210 shown in FIG. 3. As mentioned previously, because this first stimulation site 230a is on the same side of the body as the first region 210, it may be referred to as being “ipsilateral” to the first region 210. As the first region 210 in the left hemisphere 202a of the brain 200 controls movement on the right side of the body, this first potential stimulation site 230a also may be said to be contralateral to the body function impaired by the inactive status of the first region 210. The second potential stimulation site 230b, in contrast, is in the right hemisphere 202b of the brain 200 and is therefore contralateral to the first region 210 and ipsilateral to the impaired body function associated with the first region 210.

Another embodiment of process portion 102 can include generating the intended neural activity remotely from the first region 210 of the brain 200, and then detecting or sensing the location(s) in the brain where the intended neural activity has been generated. The intended neural activity can be generated by causing a signal to be generated within and/or sent to the brain. For example, in the case of a patient having an impaired limb, the affected limb is moved and/or stimulated while the brain is scanned using a known imaging technique that can detect neural activity (e.g., fMRI, PET, etc.). In one specific embodiment, the affected limb can be moved by a practitioner or the patient, stimulated by sensory tests (e.g., pricking), or subjected to peripheral electrical stimulation. In another embodiment, the patient can attempt to move the affected limb, or imagine or visualize moving the affected limb in one or more manners. The attempted or imagined movement/actual movement/stimulation of the affected limb produces a neural signal corresponding to the limb (e.g., a peripheral neural signal) that is expected to generate a response neural activity in the brain. The location(s) in the brain where this response neural activity is present can be identified using the imaging technique. By generating an intended neural activity in such a manner, this embodiment may accurately identify where the brain has recruited matter (e.g., sites 220 of FIG. 4A and/or sites 230a and 230b of FIG. 5) to perform the intended neural activity associated with the neural function.

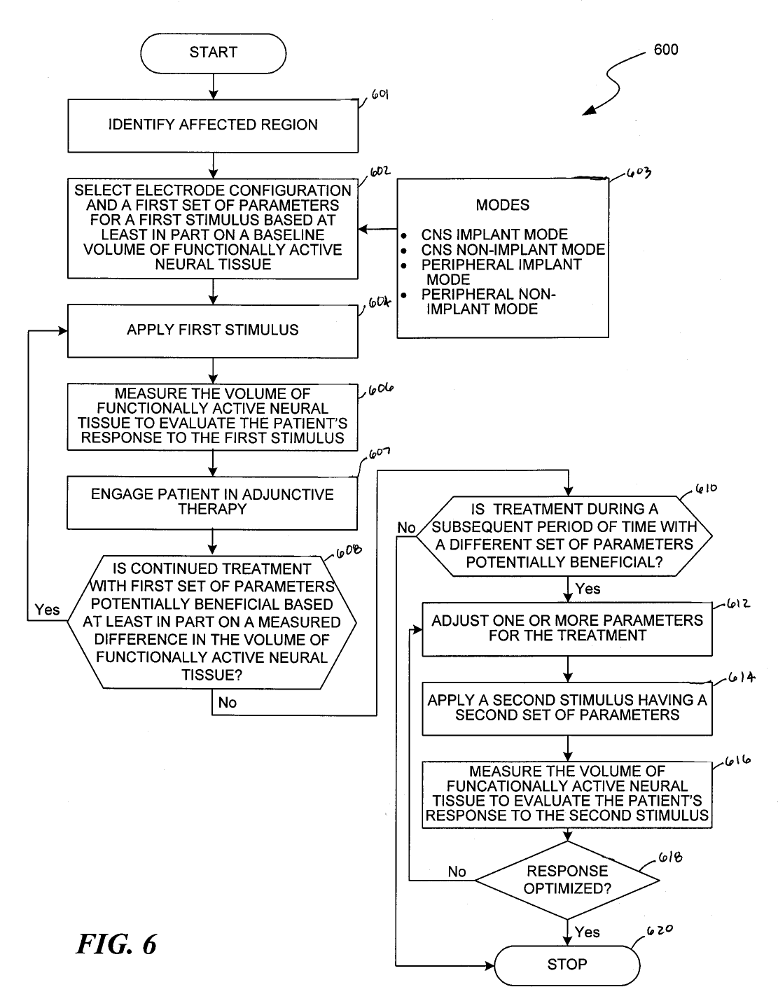

The method described above with reference to FIG. 2 is directed generally to using functional and/or structural responses obtained from stimulating a neural population with a first set of stimulation parameters to determine a second set of stimulation parameters. FIG. 6 is a flow diagram illustrating a more specific application of such a method. The process 600 shown in FIG. 6, for example, can include identifying an affected region in process portion 601. This procedure can include, for example, identifying or estimating the location of a target neural population at, proximate to, or otherwise associated with one or more particular functional areas of the patient's brain. In one particular example, a patient may have one or more areas of activation/deactivation in a particular portion of the brain, e.g., a stroke patient may have fMRI activation in ipsilesional primary motor cortex (M1), contralesional M1, and a supplementary motor area (SMA). Some or all of these areas could be identified and targeted for stimulation.

The process 600 also includes a setup procedure (process portion 602) in which an electrode configuration and the first or initial parameters for the first stimulus are selected for a first therapy sequence. The electrode configuration and first parameters can be selected based on one or more functional measurements such as, for example, the results of a preliminary fMRI scan that indicates a baseline volume of functionally active neural tissue within the patient's central nervous system (e.g., the brain, including the cerebrum, cerebral cortex, cerebellum, cerebellar cortex, deep brain structures, brain stem, and spinal column). The fMRI scan can also be used to identify one or more target neural populations for therapy. In other embodiments, other suitable methods or techniques can be used in addition to, or in lieu of, the fMRI scan to generate the baseline information.

The first parameters (as well as the particular electrode configuration) can include parameters associated with the manner in which electrical or magnetic (collectively, electromagnetic) signals are applied to the patient. Four representative modes, for example, are shown in block 603 as (a)a central nervous system (CNS) implant mode, (b) a CNS non-implant mode, (c) a peripheral implant mode, and (d) a peripheral non-implant mode. CNS modes include modes in which electromagnetic signals are provided to the patient's central nervous system. Peripheral modes include modes in which electromagnetic signals are provided to the patient's peripheral nervous system (e.g., cranial nerves (including the vagal nerve), sensory nerves, and other non-CNS nerves). Implant or invasive modes include modes in which the electromagnetic signals are delivered from a device implanted in the patient (e.g., an implanted electrode or microstimulator). Non-implant or non-invasive modes include modes in which the electromagnetic signals are delivered from a signal delivery device that is not implanted. In one embodiment, for example, an initial screening procedure may determine that a non-invasive therapy (e.g., TMS and/or tDCS) may be suitable for the patient during one or more therapy sequences. As discussed below, if the desired results are not achieved with the selected method, one or more additional non-invasive and/or invasive methods (e.g., implanted cortical stimulation), may be used.

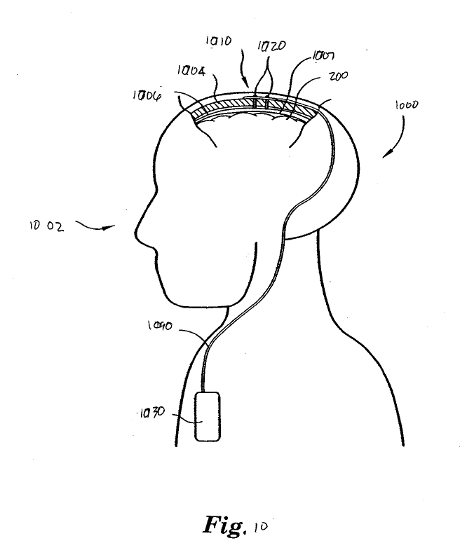

Each of the modes includes directing an application of electromagnetic signals, which can be performed automatically by an appropriately programmed computer readable medium, and/or with patient and/or practitioner involvement in a manual or semi-autonomous arrangement. The signal parameters can include signal frequency, voltage, current, and other stimulation delivery parameters. Signals can be provided to the patient in a number of different ways (e.g., individually, concurrently, serially, etc.) with one or more different stimulation modes with one or more different stimulation modes during the first therapy sequence and/or during subsequent therapy sequences. Further details of devices that provide electromagnetic signals in accordance with these modes are described below with reference to FIGS. 10-14.

The selectable signal parameters can also include the location(s) at which signals are applied. For example, the signals may be applied to different sites of the patient's nervous system during different phases of a treatment regimen. Returning to the specific example described above (process portion 601), the activation in contralesional M1 might disappear after a period of stimulation during one or more therapy sequences. At this point, stimulation to this area (which in some cases may be inhibiting functional recovery) can be discontinued, while stimulation to ipsilesional M1 would continue. In another particular example, if a large area of the patient's brain is targeted for stimulation during a particular therapy sequence and, as a result of therapy, the activated portion of the brain is consolidated or decreased in size (e.g., as determined by one or more functional measurements), the electrode contact(s) located proximate to corresponding portions of the target area that are no longer “active” could be turned off, and additional stimulation signals could only be directed to electrode contacts over the remaining active tissue in one or more subsequent therapy sequences.

In still another particular example, if an area of activation in a patient's brain in which consolidation is generally not expected (e.g., an area of hyperactivity in a tinnitus patient) becomes undesirably more or less active over time after stimulation during one or more therapy sequences, then the stimulation parameters could be adjusted to decrease or increase activation/consolidation in order to improve or reestablish therapeutic efficacy. This habituation, and the corresponding reduction or alleviation in response to modification of neural stimulation parameters, could be monitored using some measurement of neural activity (e.g., EEG, blood flow changes measured using fMRI or other suitable optical methods, MEG, SPECT, PET, MR spectroscopy, etc.).

After performing the setup procedure 602, the process 600 continues with a first stimulating procedure (process portion 604) in which the patient is treated by directing an application of electromagnetic signals to the patient during a first period of time in accordance with the first set of parameters. Depending upon embodiment details or patient condition, stimulation therapy in accordance with a particular mode or set of modes may be provided over a limited duration time period (e.g., the first therapy sequence), and stimulation therapy in accordance with a different mode or mode set may be provided over another limited duration time period or an ongoing or essentially permanent time period (e.g., a second or other subsequent therapy sequences). The signals can be provided over the course of hours, weeks, and/or months in accordance with any of several schedules. For example, the electromagnetic signals can be applied during the first therapy sequence for three hours per day, 3-5 days per week, for 2-8 or 3-6 weeks, etc., via non-implanted and/or implanted devices. The electromagnetic stimulation portion of the treatment may then be suspended for an intermediate period of time (e.g., several hours, days, weeks, or months) during which the patient may rest or consolidate neurofunctional gains, and/or still undergo adjunctive therapies. The patient may then undergo another stimulation therapy in accordance with another mode (e.g., via tDCS) for a period of hours, days or weeks (e.g., one hour, twice a week for four weeks) during the second therapy sequence

The stimulation provided during a second (and one or more additional) therapy sequences may not require implanting new electrodes, even if the electrodes implanted for stimulation during the first period of time are not positioned properly for stimulation during the first therapy sequence. For example, as discussed above, stimulation provided during the first therapy sequence may include tDCS and/or TMS stimulation. In some cases, these methods may be conducted without regard to the location of particular implanted electrodes. In other cases, it may be advantageous to provide tDCS and/or TMS in locations where electrodes have been implanted, for example, if the presence of the electrodes enhances stimulation to adjacent neural tissue even when electrical current is not provided directly (e.g., via wires) to the electrodes. In still another embodiment, the order in which the signals are applied can be reversed. For example, the signals can be provided transcranially without implanting electrodes during the first therapy sequence and then electrodes can be implanted prior to applying signals during the second therapy sequence. In any of these embodiments, the signal delivery device used to provide the electromagnetic signals may be changed from one time period to the other as part of changing from one mode to another. (e.g., by changing from implanted electrodes to a transcranial magnetic device). In further embodiments, the signal delivery device selected for a particular time period can include other devices, such as a deep brain electrode.

In process portion 607, an optional adjunctive therapy is administered to the patient. The adjunctive therapy can form a portion of the overall treatment regimen, but need not be conducted simultaneously with the administration of electromagnetic signals to the target neural population. For example, the patient may undergo a treatment session during which electromagnetic signals are applied to the target neural population, and may subsequently undergo an adjunctive therapy session that can include a motor task (e.g., a speech task, or motion of a limb), administration of drugs, and/or other type of adjunctive treatment. In terms of physical therapy, such activities can include grasping and releasing objects, stacking objects, placing objects in a box, manipulating objects, or other tasks that form part of a systematized physical therapy regimen. The nature of the task can be selected depending upon the particular condition(s) the patient is suffering from. In some embodiments, the patient can engage in adjunctive therapy simultaneously with receiving electromagnetic signals.

A response in the patient to the first stimulating procedure is detected and evaluated in a first evaluation procedure at process portion 606. The first evaluation procedure, for example, can include measuring the extent of the patient's recovery and/or one or more functional or structural features. This measurement can be made by having the patient perform tests or undergo other diagnostic procedures, in most cases, similar or identical to diagnostic procedures the patient performed before initiating the program in process portion 602. In one embodiment, for example, this process can include measuring the volume of functionally activate neural tissue after the first stimulating procedure and determining if the volume has decreased as compared with the patient's baseline volume of activation, thereby indicating that consolidation has occurred. By comparing the results after the patient has completed treatment for the first therapy sequence with results obtained either before treatment or during treatment, a practitioner can identify the progress the patient has made. The practitioner can then review the available alternate modes and select one or more modes expected to provide an enhanced effect when applied during the subsequent therapy sequence.

In process portion 608, a determination is made as to whether continued treatment in accordance with the current mode (e.g., the first set of parameters) is potentially beneficial. For example, a measured difference in the volume of functionally active neural tissue (i.e., consolidation) in the cortex and/or another portion of the patient's central nervous system (e.g. the spinal cord) during the first therapy sequence could be used to evaluate progress and, if necessary, update or optimize the signal delivery parameters (in process portion 612 described below). As mentioned previously, the existence and/or level of consolidation can be an indication of a patient's response to a given type of therapy with a particular set of parameters. Furthermore, it is expected that functional gains in patients may be longer lasting if consolidation occurs. In other embodiments, the evaluation procedure can also include measurements/analysis of other functional and/or structural features. In one embodiment, for example, diffusion tracts could be monitored and areas or regions of increased fiber density resulting from therapy (e.g., in a stroke or TBI patient) could be targeted for focused/additional/other electrical stimulation (e.g., in the context of a large electrode array with addressable contacts). In this way, the targeted stimulation site could be fine-tuned after a first or subsequent therapy sequence (e.g., a larger area of cortex would receive therapy during a first period and, after evaluation, a smaller, more focused portion of the cortex would be targeted for therapy during a second period).

In still another embodiment, one or more functional measurements can be monitored throughout stimulation and the particular stimulation parameters can be modified based on such measurements. For example, the onset of a headache in a patient during treatment may be preceded by an increase in neural activity in a given area. A sensor (e.g. a near infrared probe measuring blood flow in the occipital cortex) may detect this change in activity and trigger electrical stimulation of the occipital cortex until the targeted neural activity abates.

If the evaluation process 606 detects consolidation, then the process can return to process portion 604 for additional treatment in accordance with the first set of parameters. If a desired level of consolidation has not occurred, then in process portion 610 an evaluation is made as to whether treatment during a subsequent (e.g., second) period of time with a different set of parameters (e.g., a second set of parameters that is different than the first set of parameters, or a second mode that is different than the first mode), would be potentially beneficial. If it is determined that such a treatment would not be potentially beneficial, the treatment program is discontinued (process portion 620). For example, in some instances (e.g., stroke rehabilitation), therapy may cease to be advantageous at a particular point in time, as measured by a decrease in neural activity. This may indicate that either therapy and/or electrical stimulation should be discontinued and/or restarted after the patient's cortex has had a chance to recover.

If instead it is determined at process portion 610 that that treatment during a subsequent period of time with a different mode may be beneficial to the patient, the process 600 can further include adjusting one or more parameters of the treatment for a second therapy sequence during a subsequent period of time (process portion 612). A variety of different factors can be considered when evaluating the progress of the treatment and, subsequently, determining whether to update the treatment parameters. For example, in some cases it may be clear, based on past experience and the patient's recovery performance (e.g., the level of consolidation, etc.), in what manner the treatment program should be varied during the second therapy sequence. Such adjustments can include, for example, changing and/or modulating the location to which signals are directed, the type of signal delivery device, the signal parameters including polarity, pulse width, frequency, amplitude, etc., and/or the addition/combination of additional therapies. In addition to (or in lieu of) these factors, a number of other factors can be evaluated to determine the effectiveness of the current treatment regimen. For example, in one embodiment the effect of a second treatment modality (e.g. TMS or tDCS) on an ongoing treatment (e.g., cortical electrical stimulation) could be evaluated to optimize the signal parameters. In one particular example, in stroke patients, TMS to the contralesional hemisphere may increase the effectiveness of ipsilesional electrical cortical stimulation in producing descending volleys. In another embodiment, a localized MR spectroscopy could be used during treatment to measure the underlying metabolic activity in an area of interest.

In still another embodiment, a combination of multiple therapies (e.g. tDCS, TMS, cortical electrical stimulation) could be utilized in one or more therapy sequences. For example, an optimal current and electrode placement of tDCS could be determined using the effect of cortical electrical stimulation in activating a set of neurons by measuring the blood flow to those neurons while adjusting tDCS parameters/location. In this embodiment, multiple therapeutic parameters could be simultaneously (or approximately simultaneously) adjusted to obtain the desired functional results for the patient. In some cases, for example, this might include increased neural activity/blood flow in one area of the patient with (or without) a concomitant change in such activity in another area of the patient.

In yet another embodiment, a functional study conducted during stimulation may provide information about a distributed network that can then be targeted. For example, PET during TMS to the mirror neuron system may show areas of relative hyper- or hypoperfusion in an extended neural network connected to the mirror neurons via long- and short-distance intracortical connections. This distributed network could then be targeted with TMS and/or other types of stimulation to enhance or depress activity in particular portions of this network at one or more subsequent time periods.

In still yet another embodiment, a real time measurement could be used to determine stimulation parameters continuously during therapy. For example, ECoG could be used to measure the activity level or firing rate of a certain set of neurons. Stimulation parameters could be continuously adjusted to achieve a desired firing rate. Alternatively, near infrared light could be used to measure blood flow or deoxyhemoglobin concentration, and one or more stimulation parameters could be adjusted (as necessary) to maintain a given flow rate (e.g., deoxyhemoglobin concentration, etc.) In a further embodiment, one or more stimulation parameters could be adjusted to achieve a desired TMS-evoked MEP amplitude. The TMS-evoked MEP could be repeated daily, weekly, or at other selected time intervals to optimize the corresponding stimulation parameters.

The process can then move to process portion 614, which includes applying a second application of electromagnetic signals having a second set of parameters during the subsequent period of time (e.g., a second therapy sequence). A response in the patient to the second stimulus is then detected and evaluated in a second evaluation procedure at process portion 616. The second evaluation procedure can be generally similar to the first evaluation procedure (process portion 606) described above in which the responses are evaluated to determine specific values for the stimulus parameters that provide an efficacious result. The second evaluation procedure 616 can include, for example, again measuring the extent of functional consolidation in the patient. The evaluation procedure 616 also includes a determination routine 618 that determines whether one or more therapy sequences are appropriate. If not, (for example, if the analysis completed in process portion 616 indicated that such treatment would not be beneficial), the program is discontinued (process portion 620). If subsequent treatment would be beneficial, then the process can continue by repeating procedures 612-618 any number of times until a desirable result is achieved.

In particular embodiments, at least some of the process portions described above with reference to FIG. 6 can be automated, for example, in the context of computer-based instructions that may be resident on computer-readable media. The -computer-readable media (or aspects thereof) can be included in the devices described above, and/or in separate units. In a particular embodiment, such a computer-readable medium can include a receiver portion that is configured to receive information corresponding to neuronal structures, based on diffusion tensor imaging techniques. The computer-readable medium can further include a processor portion that is coupled to the receiver portion and is configured to evaluate one or more functional and/or structural features. The process portion can further be configured to select one or more signal delivery parameters based, at least in part, on the information. Accordingly, a computer-readable medium having the foregoing characteristics can automatically select signal delivery parameters based on one or more measurements of functional activity, with or without user intervention.

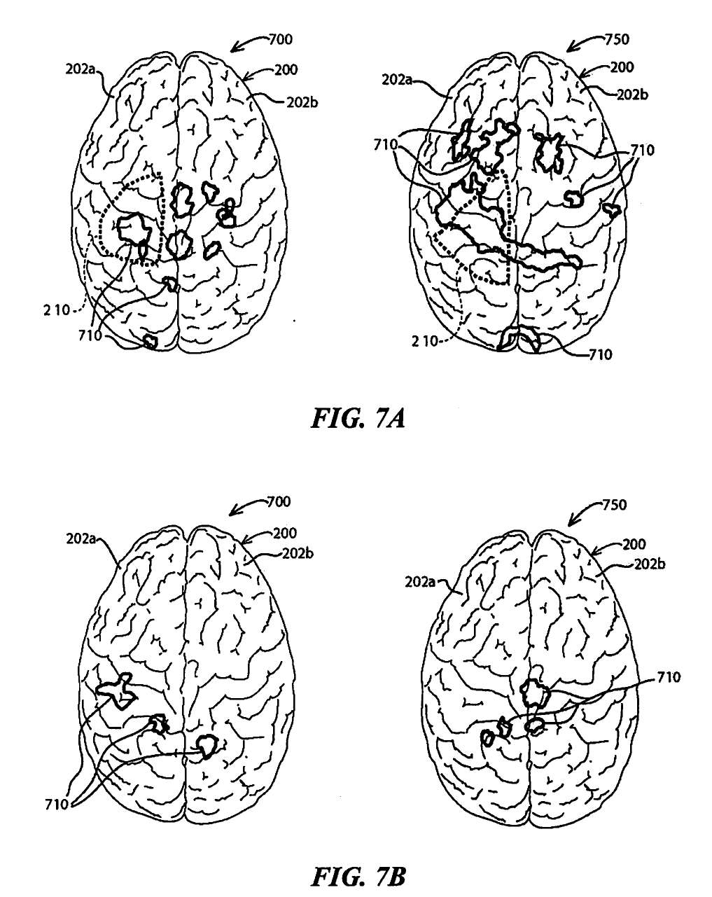

FIGS. 7A-9B illustrate several specific representative examples of treatment using one or more of the methods described above that utilize functional consolidation as an indicator of increased brain normalcy and, in some cases, favorable recovery. FIG. 7A, for example, is a top plan image of a first brain map 700 and a second brain map 750 illustrating a loss of neural activity associated with a particular neural function in a group of patients, and FIG. 7B is a top plan image of the first and second brain maps 700 and 750 illustrating the brain maps 700 and 750 after one or more therapy sequences in accordance with one particular embodiment of the invention. For a detailed overview of the study that formed the basis for the Figures shown in this particular embodiment, see “Use of Functional MRI to Guide Decisions in a Clinical Stroke Trial,” Cramer et al., Stroke, May 2005, e50-e52. This article is incorporated herein by reference in its entirety.

Referring first to FIG. 7A, the first and second brain maps 700 and 750 are representative pre-therapy images (e.g., group fMRI maps) of brains 200 from a number of patients. The first and second brain maps 700 and 750 each include first regions 210 (described in detail above with reference to FIGS. 3-5B; currently shown in broken lines) that have been affected (e.g., from a stroke, trauma, or other cause), and subsequently suffered a loss of neural activity associated with one or more particular neural functions. The first brain map 700 is based on a first group of patients (e.g., a control group including four patients), and the second brain map 750 is based on a second group of patients (e.g., an investigational group including four patients). The images shown in the first and second brain maps 700 and 750 can be created, for example, by having the patient move, attempt to move, or visualize the movement of his or her affected fingers, and then noting where neural activity occurs in response.

As shown in FIG. 7A, the neural activity that controlled the neural function for moving the fingers of the right hand no longer occurs exclusively in the first region 210. Rather, the neural function or “activation” (as shown by activated sites or regions 710) is dispersed, scattered, or otherwise unconsolidated in both the first brain map 700 and the second brain map 750. The first region 210 is thus “inactive,” which is expected to result in a corresponding loss of the movement and/or sensation in the fingers. In some instances, the damage to the patient's brain 200 may result in only a partial loss of the neural activity in the damaged region. In either case, the images of the first and second brain maps 700 and 750 shown in FIG. 7A establish that the loss of the neural function is related to the diminished neural activity in the first region 210. The brain 200 may accordingly recruit other neurons to perform neural activity for the affected neural function (e.g., via neuroplasticity), or the neural activity may not be present at any location in the brain. By generating an intended neural activity in such a manner, this embodiment may accurately identify where the brain has recruited matter (e.g., sites 710) to perform the intended neural activity associated with the neural function.

As mentioned above, FIG. 7B is a top plan image of the first and second brain maps 700 and 750 after the corresponding patients have undergone one or more therapy sequences including physical therapy and, in some cases, electrically and/or magnetically stimulating the individual patient's brains at one or more stimulation sites in accordance with the processes described above. More specifically, the post-therapy first brain map 700 illustrated in FIG. 7B is based on images from the control group of patients after a therapy sequence including only physical therapy (e.g., a three-week protocol including index finger tapping by each patient). The control group patients did not receive any electrical or magnetic stimulation. As shown in FIG. 7B, the post-therapy first brain map 700 shows little or no reduction in the volume of activation as compared with the pre-therapy first brain map 700 shown in FIG. 7A.

In contrast with the control group patients, the patients in the investigational group received targeted electrical and/or magnetic stimulation (e.g., targeted subthreshold cortical stimulation) in addition to the above-described physical therapy. As shown by the second brain map 750 in FIG. 7B, the investigational group patients had a significantly reduced volume of functional activation after treatment. This consolidation in the investigational group resembles events seen during spontaneous recovery from stroke. As discussed above, such consolidation can be an indication of a patient's response to therapy. Furthermore, it is expected that functional gains in patients may be longer lasting if consolidation occurs. In contrast, continued activation in the targeted areas or larger areas or regions of activation in the targeted areas or other areas of the patient's central nervous system can be an indication of non-response to the therapy. Accordingly, as discussed previously, consolidation can be used as an indicator to select patients with whom to continue a given type of therapy (“responders”) in contrast with patients who should have therapy modified, receive a different type of therapy, or stop therapy altogether (“non-responders”).

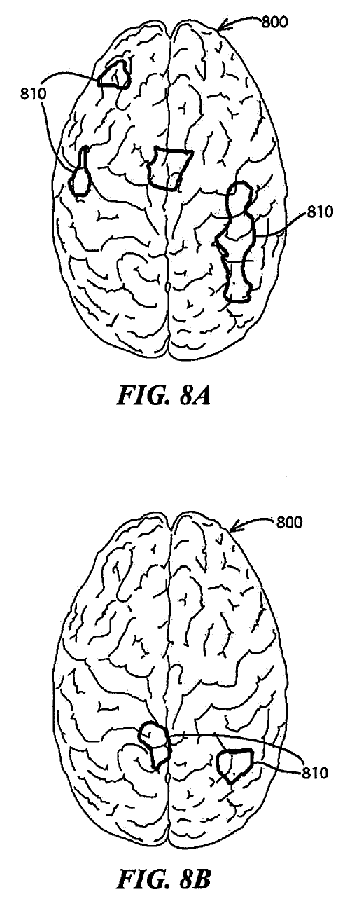

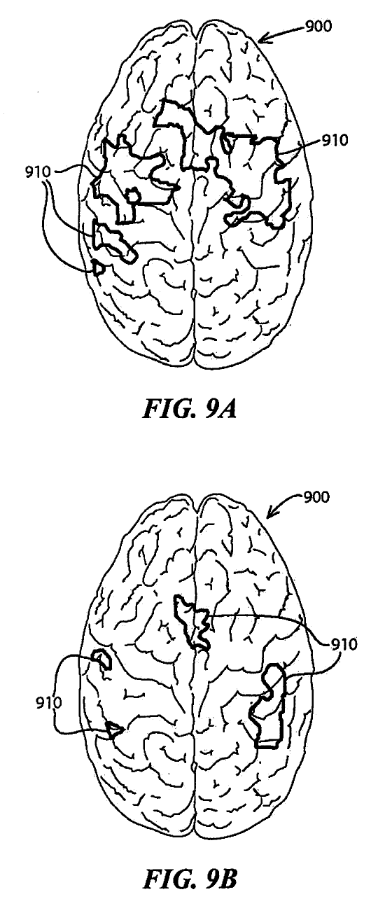

FIGS. 8A-9B illustrate two more specific examples of treatment using one or more of the methods described above. FIG. 8A, for example, is a pre-therapy, top plan image of a patient's brain 800 after one or more regions within the brain have been affected by a stroke. The neural activity that controlled one or more particular neural functions of the patient no longer occurs in a particular area or region, and instead is dispersed and/or scattered about a number of various regions of the brain 800 (as shown by activated sites 810). In this particular embodiment, for example, the total volume of the activated sites 810 is about 9,564 mm³. The total volume can be determined using, for example, suitable functional neuroimaging and signal processing techniques. FIG. 8B is a post-therapy, top plan image of the brain 800 after one or more therapy sequences in accordance with the methods described above. As illustrated, the total volume of activation within the brain 800 is significantly less after the therapy sequence(s). In this particular embodiment, for example, the activation volume after therapy is about 2,554 mm³. This significant consolidation can accordingly be indicative of the patient's response to therapy.

FIG. 9A is a pre-therapy, top plan image of a patient's brain 900 after one or more regions within the brain have been affected from a stroke in accordance with still another embodiment. The brain 900 has also been affected by a stroke, and includes a number of activated sites 910. The brain 900 has a pre-therapy activation volume of approximately 17,646 mm³. Referring next to FIG. 9B, after one or more therapy sequences in accordance with the methods described above, the post-therapy activation volume has decreased to approximately 6,917 mm³.

### C. Applying Electrical Stimulation to a Patient and Techniques for Detecting Responses to Such Stimulation

FIGS. 10-14 illustrate representative devices for applying electrical signals. These devices, for example, can be located at a signal delivery site to provide the first signal having the first set of stimulation parameters (as described above with reference to FIGS. 2 and 6). Once the second set of stimulation parameters is determined, the same or similar devices can provide the second stimulation having the second set of stimulation parameters. FIG. 10 is a schematic illustration of a neurostimulation system 1000 implanted in a patient 1002 to provide stimulation in accordance with several embodiments of the invention. The system 1000 can include an electrode device 1010 carrying one or more electrodes 1020. The electrode device 1010 can be positioned in the skull 1004 of the patient 1002, with the electrodes 1020 positioned to stimulate target areas of the brain 200. For example, the electrodes 1020 can be positioned just outside the dura mater 1006 (which surrounds the brain 200) to stimulate cortical tissue. In another embodiment described later with reference to FIG. 11, an electrode can penetrate the dura mater 1006 to stimulate subcortical tissues. In still further embodiments, the electrodes 1020 can penetrate the dura mater 1006 but not the underlying pia mater 1007, and can accordingly provide stimulation signals through the pia mater 1007.

The electrode device 1010 can be coupled to a pulse system 1030 with a communication link 1090. The communication link 1090 can include one or more leads, depending (for example) upon the number of electrodes 1020 carried by the electrode device 1010. The pulse system 1030 can direct electrical signals to the electrode device 1010 to stimulate target neural tissues.

The pulse system 1030 can be implanted at a subclavicular location, as shown in FIG. 10. In particular embodiments, the pulse system 1030 (and/or other implanted components of the system 1000) can include titanium and/or other materials that can be exposed to magnetic fields generated by magnetic resonance chambers without harming the patient. The pulse system 1030 can also be controlled internally via pre-programmed instructions that allow the pulse system 1030 to operate autonomously after implantation. In other embodiments, the pulse system 1030 can be implanted at other locations, and at least some aspects of the pulse system 1030 can be controlled externally. For example, FIG. 8 illustrates an embodiment of the system 1000 in which the pulse system 1030 is positioned on the external surface of the skull 1004, beneath the scalp 1005. The pulse system 1030 can be controlled internally and/or via an external controller 1035.

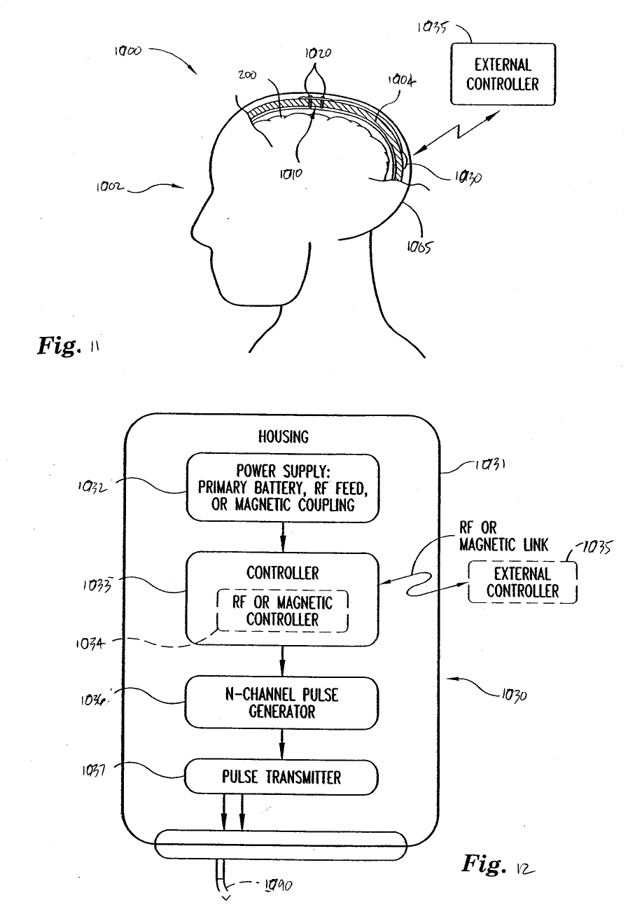

FIG. 12 schematically illustrates a representative example of a pulse system 1030 suitable for use in the neural stimulation system 1000 described above. The pulse system 1030 generally includes a housing 1031 carrying a power supply 1032, an integrated controller 1033, a pulse generator 1036, and a pulse transmitter 1037. The power supply 1032 can be a primary battery, such as a rechargeable battery or other suitable device for storing electrical energy. In other embodiments, the power supply 1032 can be an RF transducer or a magnetic transducer that receives broadcast energy emitted from an external power source and that converts the broadcast energy into power for the electrical components of the pulse system 1030.

In one embodiment, the integrated controller 1033 can include a processor, a memory, and a programmable computer medium. The integrated controller 1033, for example, can be a microcomputer, and the programmable computer medium can include software loaded into the memory of the computer, and/or hardware that performs the requisite control functions. In another embodiment identified by dashed lines in FIG. 12, the integrated controller 1033 can include an integrated RF or magnetic controller 1034 that communicates with the external controller 1035 via an RF or magnetic link. In such an embodiment, many of the functions performed by the integrated controller 1033 may be resident on the external controller 1035 and the integrated portion 1034 of the integrated controller 1033 may include a wireless communication system.

The integrated controller 1033 is operatively coupled to, and provides control signals to, the pulse generator 1036, which may include a plurality of channels that send appropriate electrical pulses to the pulse transmitter 1037. The pulse generator 1036 may have multiple channels, with at least one channel associated with a particular one of the electrodes 1020 described above. The pulse generator 1036 sends appropriate electrical pulses to the pulse transmitter 1037, which is coupled to the electrodes 1020 (FIG. 10). In one embodiment, each of these electrodes 1020 is configured to be physically connected to a separate lead, allowing each electrode 1020 to communicate with the pulse generator 1036 via a dedicated channel. Suitable components for the power supply 1032, the integrated controller 1033, the external controller 1035, the pulse generator 1036, and the pulse transmitter 1037 are known to persons skilled in the art of implantable medical devices.

The pulse system 1030 can be programmed and operated to adjust a wide variety of stimulation parameters, for example, which electrodes are active and inactive, whether electrical stimulation is provided in a unipolar or bipolar manner, and/or how the stimulation signals are varied. In particular embodiments, the pulse system 1030 can be used to control the polarity, frequency, duty cycle, amplitude, and/or spatial and/or temporal qualities of the stimulation. The stimulation can be varied to match naturally occurring burst patterns (e.g., theta burst stimulation), and/or the stimulation can be varied in a predetermined, pseudorandom, and/or aperiodic manner at one or more times and/or locations.

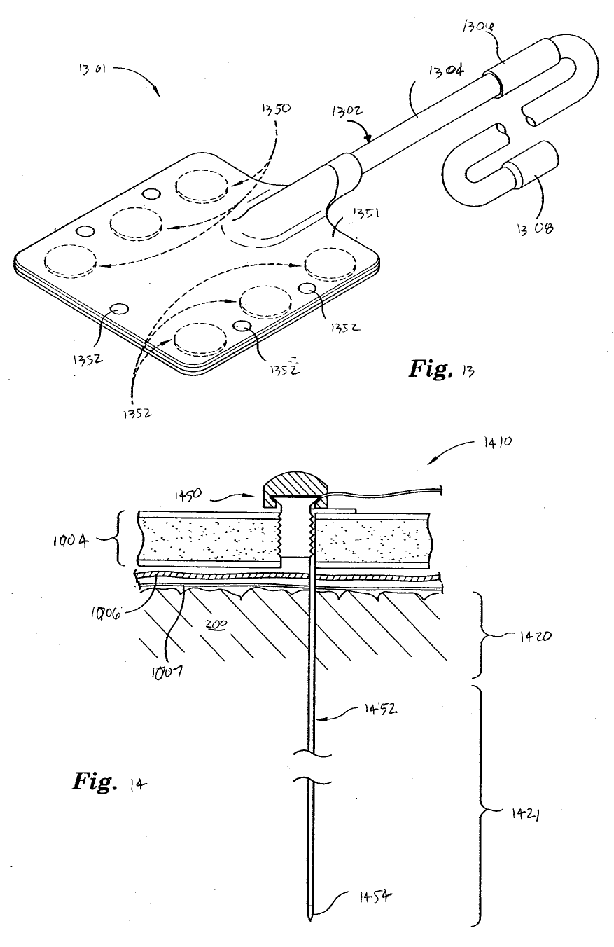

Stimulation can be provided to the patient using devices in addition to or in lieu of those described above. For example, FIG. 13 is a top, partially hidden isometric view of an embodiment of an electrode device 1301 configured to carry multiple cortical electrodes 1350. The electrodes 1350 can be carried by a flexible support member 1351 (located within the patient's skull) to place each electrode 1350 at a stimulation site of the patient when the support member 1351 is implanted within the patient's skull. Electrical signals can be transmitted to the electrodes 1350 via leads carried in a communication link 1302. The communication link 1302 can include a cable 1004 that is connected to the pulse system 1030 (FIG. 12) via a connector 1306, and is protected with a protective sleeve 1308. Coupling apertures or holes 1352 can facilitate temporary attachment of the electrode device 1301 to the dura mater at, or at least proximate to, a stimulation site. The electrodes 1350 can be biased cathodally and/or anodally, as described above. In an embodiment shown in FIG. 13, the electrode device 1301 can include six electrodes 1350 arranged in a 2×3 electrode array (i.e., two rows of three electrodes each), and in other embodiments, the electrode device 1301 can include more or fewer electrodes 1350 arranged in symmetrical or asymmetrical arrays. The particular arrangement of electrodes 1350 can be selected based on the region of the patient's brain that is to be stimulated, and/or the patient's condition.

FIG. 14 illustrates an electrode device 1401 that may be configured to apply electrical stimulation signals to a cortical region 1420 or a subcortical region 1421 of the brain 200 in accordance with further embodiments of the invention. The electrode device 1401 can include an electrode 1450 having a head and a threaded shaft that extends through a pilot hole in the patient's skull 1004. If the electrode 1450 is intended for cortical stimulation, it can extend through the skull 1004 to contact the dura mater 1006 or the pia mater 1007. If the electrode 1450 is to be used for subcortical stimulation, it can include an elongate conductive member 1452 that extends downwardly through the cortical region 1036 into the subcortical region 1037. Most of the length of the elongate conductive member 1054 can be insulated, with just a tip 1454 exposed to provide electrical stimulation in only the subcortical region 1037. Subcortical stimulation may be appropriate in at least in some instances, for example, when the brain structures such as the basal ganglia are to be stimulated. In other embodiments, other deep brain structures (e.g., the amygdala or the hippocampus) can be stimulated using a subcortical electrode. If the hippocampus is to be stimulated, stimulation may be provided to the perihippocampal cortex using a subdurally implanted electrode that need not penetrate through brain structures other than the dura.

Further details of electrode devices that may be suitable for electromagnetic stimulation in accordance with other embodiments of the invention are described in the following pending U.S. patent applications, all of which are incorporated herein by reference: Ser. No. 10/891,834, filed Jul. 15, 2004; Ser. No. 10/418,796, filed Apr. 18, 2003; and Ser. No. 09/802,898, filed Mar. 8, 2001. Further devices and related methods are described in a copending U.S. patent application Ser. No. 11/255,187, entitled “Systems and Methods for Patient Interactive Neural Stimulation and/or Chemical Substance Delivery,” and U.S. patent application Ser. No. 11/254,060, entitled “Methods and Systems for Improving Neural Functioning, Including Cognitive Functioning and Neglect Disorders,” both of which are incorporated herein by reference.

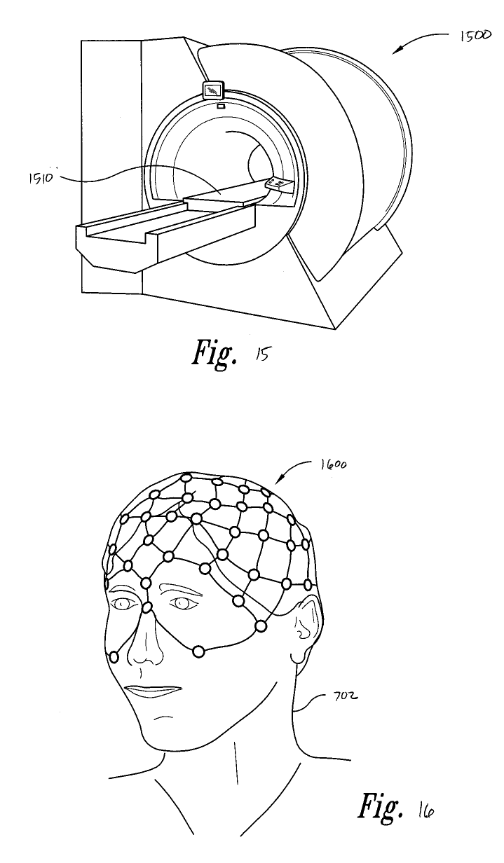

Once the appropriate signal delivery device has been selected and positioned, the practitioner can apply signals and, particularly if the practitioner is stimulating the target neural population, detect a response. The practitioner may also wish to detect a response when stimulation is applied during a subsequent therapy sequence, e.g., to verify that the stimulation provided in accordance with the second set of stimulation parameters is or appears to be producing a desired response, condition, state, or change. In a particular aspect of either process, the response is detected at least proximate to the patient's central nervous system, and in a further particular aspect, at the patient's brain. One or more of several techniques may be employed to determine the neural response to the stimulation. Many suitable techniques rely on hemodynamic properties, e.g., they measure or are based on concentrations of oxy-hemoglobin and/or deoxy-hemoglobin. Such techniques can include fMRI, measurements or estimates of cerebral blood flow, cerebral blood volume, cerebral metabolic rate of oxygen (CMRO), Doppler flowmetry, and/or optical spectroscopy using near infrared radiation. Magnetic resonance techniques (e.g., fMRI techniques) can be performed inside a magnetic resonance chamber, as described later with reference to FIG. 15

Certain other techniques, e.g., thermal measurements and/or flowmetry techniques, can be performed subdermally on the patient. Still further techniques, in particular, optical techniques such as near infrared spectroscopy techniques, are generally noninvasive and do not require penetration of the patience's scalp or skull. These techniques can include placing a near infrared emitter and detector (or an array of emitter/detector pairs) on the patient's scalp to determine species concentrations of both oxy-hemoglobin and deoxy-hemoglobin. Representative devices for measuring hemodynamic quantities (that correspond to neural activity) are disclosed in U.S. Pat. No. 5,024,226 and U.S. Pat. No. 6,615,065, both of which are incorporated herein by reference, and are available from ISS, Inc. of Champaign, Ill., and Somanetics of Troy, Mich. Further devices and associated methods are disclosed in pending U.S. patent application Ser. No. 11/583,349 entitled “Neural Stimulation and Optical Monitoring Systems and Methods,” and incorporated herein by reference. Any of the foregoing techniques can be used to identify and/or quantify parameters and/or states associated with the patient's level of neural functioning. Such states may determine, influence, and/or alter signal properties such as intensity, power, spectral, phase, coherence, and/or other signal characteristics.

FIG. 15 illustrates a magnetic resonance imaging system 1500 having a patient platform 1510 for carrying the patient during a procedure for detecting responses to stimulation. Functional MRI techniques can be used to correlate levels of brain activity with stimulation provided to the patient's brain via one or more stimulation parameters. If the stimulation is to be provided via implanted devices, the implanted devices are selected to be compatible with the strong magnetic fields generated by the chamber.

Some embodiments of the invention may involve magnetic resonance spectroscopy (MRS) techniques, which may facilitate the identification or determination of various chemical species and/or relative concentration relationships between such species in particular brain regions. Stimulation sites may be selected based upon, for example, a detected imbalance between particular neurotransmitters. Additionally or alternatively, the effect(s) of neural stimulation may be evaluated or monitored on a generally immediate, short term, and/or long term basis using MRS and/or other imaging techniques.

FIG. 16 illustrates a patient wearing an electrode net 1600 that includes a network of receptor electrodes positioned over the patient's scalp to sense, detect, or measure EEG signals corresponding to the patient's neuroelectric activity. In a representative embodiment, the electrode net 1600 may include a Geodesic Sensor Net manufactured by Electrical Geodesics, Inc., of Eugene, Oreg. When external or non-intrinsic electromagnetic stimulation generates or affects a locational, spectral, and/or temporal response or change in the patient's neuroelectric activity, such responses or changes in the patient's neuroelectric signals can be sensed or detected by the electrode net 1300. Accordingly, the detected properties of or changes in neuroelectric signals (or the relative absence of particular characteristics or changes) can be used to determine whether the threshold level for a target neural population has been met. In particular embodiments, the foregoing sensors can provide coherence information, which relates to the rhythmic or synchronous aspects of the patient's neural activity. Further details regarding coherence are disclosed in co-pending U.S. patent application Ser. No. 10/782,526, filed on Feb. 19, 2004, and incorporated herein by reference.

In other embodiments, a net (or other network) generally similar to that shown in FIG. 16 can be outfitted with sensors other than electrical sensors. For example, such a net can be outfitted with near infrared sensors or other optical sensors. Such sensors may detect changes in neural activity arising in association with subthreshold, threshold, and/or suprathreshold level electromagnetic stimulation.

From the foregoing, it will be appreciated that specific embodiments of the invention have been described herein for purposes of illustration, but that various modifications may be made without deviating from the invention. For example, many of the techniques described above in the context of cortical stimulation from within the skull can also be applied to cranial nerves (e.g., the vagal nerve) that may be accessible without entry directly through the patient's skull. Many of the techniques described above in the context of subthreshold stimulation may be applied as well in the context of superthreshold stimulation. Aspects of the invention described in the context of two therapy sequences and/or time periods may apply to more therapy sequences or time periods (e.g., three or more) in other embodiments. Electromagnetic signals described in some embodiments as stimulation signals may be replaced with inhibitory signals in other embodiments, for example, by changing signal frequency and/or other signal delivery parameters.

Aspects of the invention described in the context of particular embodiments may be combined or eliminated in other embodiments. For example, final placement of an electrode could be determined using an intra-operative measurement of a functional and/or structural metric. In one embodiment, for example, an intra-operative fMRI or intra-operative ECOG may be used to select a target neural population that, when stimulated, can cause signal changes in one or more particular areas of interest. Further, many of the signal delivery devices described above may have other configurations and/or capabilities in other embodiments. Further, while advantages associated with certain embodiments of the invention have been described in the context of those embodiments, other embodiments may also exhibit such advantages, and not all embodiments need necessarily exhibit such advantages to fall within the scope of the invention. Accordingly, the invention is not limited except as by the appended claims.

I/we claim:

1. A method for treating a patient, the method comprising:
measuring a volume of functionally active neural tissue within a patient's central nervous system both before and after affecting a target neural population of the patient with electromagnetic signals; and
based at least in part on a measured difference in the volume of functionally active neural tissue, controlling at least one signal delivery parameter with which the electromagnetic signals are applied to the patient.

2. The method of claim 1 wherein controlling at least one signal delivery parameter comprises changing at least one signal delivery parameter.

3. The method of claim 1 wherein controlling at least one signal delivery parameter comprises changing a location at which the stimulation is applied.

4. The method of claim 1 wherein controlling at least one signal delivery parameter comprises changing at least one of a current, voltage, and waveform of a stimulation signal applied to the patient.

5. The method of claim 1 wherein controlling at least one signal delivery parameter comprises at least one of initiating, continuing, varying interrupting, resuming, and discontinuing the application of the electromagnetic stimulation to the target neural population.

6. The method of claim 1 wherein controlling at least one signal delivery parameter comprises discontinuing treatment if the volume of functionally active neural tissue within the patient's central nervous system remains approximately constant or increases after affecting the target neural population.

7. The method of claim 1 wherein affecting a target neural population of the patient with electromagnetic stimulation comprises affecting the target neural population with a signal delivery device selected from at least one of the following (a) a non-invasive device that is not implanted in the patient's body; (b) an invasive device that includes at least one electrode implanted in the patient's body; and (c) a combination of non-invasive and invasive devices.

8. The method of claim 7 wherein controlling at least one signal delivery parameter comprises selecting a signal delivery device having a different configuration with which to affect the target neural population.

9. The method of claim 7 wherein affecting a target neural population with a non-invasive device comprises affecting the target neural population with at least one of transcranial magnetic stimulation (TMS) and transcranial direct current stimulation (tDCS).

10. The method of claim 1, further comprising directing the patient to engage in an adjunctive therapy.

11. The method of claim 10 wherein the adjunctive therapy is selected to include behavioral therapy.

12. The method of claim 10 wherein directing the patient to engage in an adjunctive therapy includes directing the patient to engage in an adjunctive therapy during a treatment session that also includes applying the electromagnetic stimulation.

13. The method of claim 1 wherein measuring a volume of functionally active neural tissue comprises using magnetic resonance (MRI) techniques.

14. The method of claim 1 wherein measuring a volume of functionally active neural tissue comprises using an electroencephalography (EEG).

15. The method of claim 1 wherein affecting a target neural population comprises applying electromagnetic stimulation from at least one electrode implanted within the patient's skull.

16. The method of claim 1 wherein affecting a target neural population comprises applying electromagnetic stimulation from a location external to the patient's skull.

17. The method of claim 1 wherein measuring a volume of functionally active neural tissue, affecting a target neural population, and controlling at least one signal delivery parameter are performed at least in part by a computer-readable medium.

18. The method of claim 1 wherein measuring a volume of functionally active neural tissue, affecting a target neural population, and controlling at least one signal delivery parameter are performed at least in part by a practitioner.

19. The method of claim 1, further comprising selecting one of more target neural populations to which the stimulation signals are provided.

20. The method of claim 19 wherein selecting one of more target neural populations comprises selecting the one or more target neural populations based, at least in part, on the regions of functionally active neural tissue within the patient's central nervous system.

21. The method of claim 1 wherein measuring a volume of functionally active neural tissue and controlling at least one signal delivery parameter occur approximately in real time.

22. The method of claim 1 wherein measuring a volume of functionally active neural tissue before stimulation, measuring a volume of functionally active neural tissue after stimulation, and controlling at least one signal delivery parameter based at least in part on the volume difference occur sequentially.

23. A method for treating a patient, the method comprising:
affecting a target neural population of the patient by providing electromagnetic signals to the target neural population;
detecting and measuring functional consolidation within the patient's central nervous system after providing the electromagnetic signals to the target neural population; and
based at least in part on the measured functional consolidation, controlling at least one signal delivery parameter in accordance with which the electromagnetic signals are applied to the target neural population.

24. The method of claim 23 wherein controlling at least one signal delivery parameter comprises changing at least one a current, voltage, and waveform of a stimulation signal applied to the patient.

25. The method of claim 23 wherein controlling at least one signal delivery parameter comprises discontinuing treatment if the volume of functional consolidation within the patient's central nervous system remains approximately constant or increases after affecting the target neural population.

26. The method of claim 23 wherein affecting a target neural population of the patient comprises affecting the target neural population with a signal delivery device selected from at least one of the following (a) a non-invasive device that is not implanted in the patient's body; (b) an invasive device that includes at least one electrode implanted in the patient's body; and (c) a combination of non-invasive and invasive devices.

27. The method of claim 23 wherein detecting and measuring functional consolidation and controlling at least one signal delivery parameter occur approximately in real time.

28. A method for treating a patient, the method comprising:
applying electromagnetic stimulation to the patient's brain to at least partially reduce a volume of functionally activated tissue within the brain;
determining a volume of functionally activated tissue using a functional MRI (fMRI) process after applying the electromagnetic stimulation; and
based at least in part on the results of the fMRI process, performing at least one of the following functions
(a) changing at least one parameter in accordance with which the electromagnetic stimulation is applied;
(b) ceasing to apply the electromagnetic stimulation; and
(c) maintaining stimulation parameters in accordance with which the electromagnetic stimulation is applied.

29. The method of claim 28 wherein changing at least one parameter in accordance with which the electromagnetic stimulation is applied comprises changing at least one of a current, voltage, and waveform of a stimulation signal applied to the patient.

30. The method of claim 28, further comprising directing the patient to engage in an adjunctive therapy during a treatment session that also includes applying the electromagnetic stimulation.

31. A method for treating a patient, the method comprising:
identifying one or more target neural populations of the patient's central nervous system associated, at least in part, with a particular motor function in the patient;
applying first electromagnetic signals to the target neural population in accordance with a first set of signal delivery parameters to produce functional consolidation within the central nervous system; and
applying second electromagnetic signals in accordance with a second set of signal delivery parameters to the corresponding one or more target neural populations, wherein the second set of signal delivery parameters are based, at least in part, on a level of functional consolidation in the patient after applying the first electromagnetic signals.

32. The method of claim 31 wherein applying second electromagnetic signals in accordance with a second set of signal delivery parameters comprises applying second electromagnetic signals having at least one of a current, a voltage, and a waveform different than the first electromagnetic signals.

33. The method of claim 31 wherein applying second electromagnetic signals in accordance with a second set of signal delivery parameters comprises applying second electromagnetic signals at a location different from a location at which the first electromagnetic signals were applied.

34. The method of claim 31, further comprising discontinuing treatment if level of functional consolidation in the patient remains approximately constant or increases after applying first electromagnetic signals to the target neural population.

35. A method for treating a patient, the method comprising:
applying a first stimulus to a target neural population of a patient associated with a particular motor function and using a first set of stimulation parameters;
measuring a response to the first stimulus at least proximate to the patient's central nervous system using a functional neuroimaging process; and
based at least in part on the results of the functional neuroimaging process, applying a second stimulus to the target neural population using a second set of stimulation parameters.

36. The method of claim 35 wherein measuring a response to the first stimulus comprises measuring a volume of functionally active neural tissue within the patient's central nervous system after applying the first stimulus to the target neural population.

37. The method of claim 35 wherein applying a second stimulus to the target neural population using a second set of stimulation parameters comprises applying a second stimulus having at least one of a current, a voltage, and a waveform different than the first stimulus.
